(19) United States
(12) Patent Application Publication
(10) Pub. No.: US 2003/0023033 A1
(43) Pub. Date: Jan. 30, 2003

(54) Novel class II cytokine receptors and uses thereof

(76) Inventors: Laure Dumoutier, Brussels (BE); Jean-Christophe Renauld, Brussels (BE)

Correspondence Address: FULBRIGHT & JAWORSKI, LLP, 666 FIFTH AVE, NEW YORK, NY, 10103-3198, US

(21) Appl. No.: 09/915,735

(22) Filed: Jul. 26, 2001

(51) Int. Cl. C07K 14/715; A61K 39/395; C07K 16/28

(52) U.S. Cl. 530/350; 530/388.22; 424/144.1

(57) ABSTRACT

A new class II cytokine receptor has been identified, which comprises an interleukin-22 receptor molecule, and an interleukin-20 receptor β molecule. The complex binds to cytokines homologous to IL-10, including mda-7 and IL-20. Also described are methods for inhibiting effect of interleukin-22 and/or interleukin-20 on cells. The latter is especially useful in treatment of, e.g., skin diseases such as psoriasis.

### FIELD OF THE INVENTION

This invention relates to cytokines, and the phenomenon of “receptor switching.” More particularly, it relates to homologs of IL-10, and various receptors for these homologs, as well as their use.

### BACKGROUND AND PRIOR ART

Interleukin-10 (“IL-10” hereafter) is a major, anti-inflammatory cytokine, which was originally identified by Moore, et al., Annu. Rev. Immunol 19:683 (2001), as a factor which inhibited cytokine production by activated TH1 lymphocytes. Following the identification of IL-10, several additional cytokines, with varying degrees of homology to IL-10 were identified. The first of these was named “mda-7”, an acronym for “melanocyte differentiation associated gene 7”, because its expression was upregulated during in vitro differentiation of a melanoma cell line. See Jiang, et al., Oncogene 11:2477 (1995). This protein exhibits 22% amino acid identity with IL-10, but it was not originally recognized as a secreted protein. Expression of mda-7 is reported to provoke irreversible growth arrest of tumors via induction of apoptosis or differentiation; however, it is not clear if this effect results from a paracrine loop that involves a classic cytokine receptor pathway, or from a cytoplasmic form of the mda-7 molecule. Recently, Schaefer, et al., J. Immunol 166:5859 (2001), identified the murine orthologue of mda-7, as a TH2-specific cytokine, and named it “IL-4 induced secreted protein,” or “FISP.” The rat counterpart, identified by Zhang, et al., J. Biol. Chem 275:24436 (2000), is referred to as “mob5”, and is expressed by intestinal epithelial cells upon ras activation. Zhang et al. have suggested that mob5 plays a role in ras oncogene-mediated neoplasia, through an autocrine loop involving a putative, ras-inducible cell surface receptor. Soo, et al., J. Cell Biochem. 74:1 (1999), have cloned the gene as a gene that is overexpressed in the skin during wound healing.

Both the IL10 and mda 7 genes have been mapped to chromosome 1q31-32, which is a region where two other, IL-10 related genes are found, i.e., “IL19” and “IL20.” IL-19 is expressed by LPS activated peripheral blood mononuclear cells, as reported by Gallagher, et al., Genes Immun 1:442 (2000). As for IL-20, its biological activities have been studied by using transgenic mice which overexpress the cytokine, where the gene is under the control of various promoters. Such mice, as reported by Blumberg, et al., Cell 104:9 (2001), are characterized by neonatal lethality, and skin abnormalities, including aberrant epidermal differentiation, which is reminiscent of psoriasis lesions in humans. Blumberg, et al., have described the IL-20 receptor complex as a heterodimer of two orphan class II cytokine receptor subunits. Specifically, “CRF2-8,” for which the name “IL-20Rα” has been suggested, and “DIRS1”, for which IL-20Rβ” has been suggested.

Two other IL-10 cytokines, i.e., “AK155” and “IL-22” are located on human chromosome 12q15, near the IFN-γ gene. AK155 is known to be upregulated by Herpes saimiri infection of T lymphocytes. See Knappe, et al., J. Virol 74:3381 (2000). The IL-22 molecule was originally described as an IL-9 inducible gene, and was referred to as “IL-TIF,” for “IL-10 related T cell derived inducible factor.” See Dumoutier, et al., J. Immunol 164:1814 (2000), incorporated by reference, as well as PCT Application WO 00/24758, incorporated by reference and the U.S. priority applications referred to therein. The activities of IL-22 include the induction of the acute phase response, especially in hepatocytes and they are mediated through a heterodimeric receptor which consists of the CRF2-9/IL22R subunit, and the β chain of the IL-10 receptor. See, e.g., Dumoutier, et al., Proc. Natl. Acad. Sci USA 97:10144 (2000); Kotenko, et al., J. Biol. Chem 276:2725 (2000); Xie, et al., J. Biol. Chem 275:31335 (2000), all of which are incorporated by reference. Induction of the acute phase response is associated with inflammation, allergic responses, and cancer, thus suggesting that modulation of the interaction between IL-9 and IL-22 can lead to alleviation of these conditions. In addition to its cellular receptor, IL-22 binds to a secreted member of the class II cytokine receptor family, referred to as “IL-22BP,” or “IL-22 binding protein,” which acts as a natural IL-22 antagonist. See Dumoutier, et al., J. Immunol 166:7090 (2001), Kotenko, et al., J. Immunol 166:7096 (2001), incorporated by reference. “IL-22R” as used hereafter refers to “IL-22R” as described in the listed papers. “IL-20Rα” and “IL-20Rα” as used hereafter refers to the terms as used supra.

It will be understood from the above, that there are two classes of cytokine receptors, i.e., class I and class II. Within the class I cytokine receptors, sharing of receptor subunits is a well recognized phenomenon. Subfamilies have been defined as a result of this phenomenon, including the βc, gp130, and IL-2Rγ families. In the case of class II receptors, however, the only example of a shared receptor up to now has been the IL-10Rβ chain, which is involved in both IL-10 and IL-22 signaling. See Dumoutier, et al., Proc. Natl. Acad. Sci USA 97:10144 (2000); Kotenko, et al., J. Biol. Chem 276:2725 (2000); Xie, et al., J. Biol. Chem 275:31335 (2000). It is of interest to determine what class II cytokine receptors can and do function in connection with binding to particular cytokines. This is one feature of the invention, which will be described together with other features of the invention, in the disclosure which follows.

### DETAILED DESCRIPTION OF PREFERRED EMBODIMENTS

### Example 1

A series of experiments were carried out in order to characterize interactions between various IL-10 homologues and receptors which belong to the class II cytokine receptor family.

To do this, coding sequences for the IL-10 homologues mda-7, IL-19, IL-20 and IL-22 were amplified via RT-PCR, using RNA from T cells which had been stimulated with anti-CD3 antibodies, using standard, art recognized techniques. The coding sequence for IL-20 was amplified from skin RNA.

The resulting cDNA molecules were then cloned into a commercially available plasmid, i.e., pCEP4, under the control of a CMV promoter. Fusion proteins, referred to as “mda-7-flag,” “IL-19-flag,” and “IL-22-flag” were produced. To elaborate, cDNA constructs were prepared by placing a sense primer in pCEP4 before the cloning site, as well as an antisense primer, in which the STOP codon of the cDNA was mutated, together with a nucleotide sequence coding for the first part of the flag molecule. The sequences used were:

gtccttgtag tcacctcccc cgagcttgta gaatttctg (SEQ ID NO: 1; used with mda-7);

gtccttgtag tcacctcccc cagctgagga catacttc (SEQ ID NO: 2; used with IL-19);

gtccttgtag tcacctcccc cttctgtctc ctccatcca (SEQ ID NO: 3; for IL-20);

and

gtccttgtag tcacctcccc cgacgcaagc atttctcag (SEQ ID NO: 4; used with IL-22).

A first PCR product was prepared, and then a second PCR amplification was carried out, using the same sense primer, as well as an oligonucleotide which contained the entire flag sequence, as well as an NheI restriction site, i.e.:

actgctagct cacttgtcgt catcgtcctt gtagtcacct (SEQ ID NO: 5).

This allowed for direct cloning into the pCEP4 plasmid.

Clones which contained the cytokine-flag fusion cDNA were sequenced, using standard techniques and a commercially available automated fluorescence based system.

The fusion proteins were then produced by transient expression in HEK293-EBNA cells. To do this, cells were seeded in 6 well plates, at 8×10⁵ cells/well, one day before transfection. Transfection was then carried out using a standard lipofectamine method, following manufacturer's instructions, using 2 μg of plasmid DNA.

Following this, the cells were incubated in 2 ml of medium, for four days, to maximize production of proteins.

Recombinant protein was measured, via Western blotting, using an antibody specific for the flag peptide, by combining 10 μl of cell supernatant with Laemmli sample buffer, followed by boiling for 5 minutes. The proteins were separated on a precast, 14% polyacrylamide gel. Following transfer, the PVDF membrane was blocked in 5% nonfat dry milk, washed and probed with biotinylated, anti-flag antibody (25 μg/ml), followed by incubation with streptavidin peroxidase (1/5000). Electrochemiluminescence was measured, using a commercially available system.

The HEK-293 cells secreted mda-7, IL-19, and IL-22-flag-fusion proteins with a heterogeneous molecular weight, ranging from 23-30 kilodaltons. The heterogeneity most likely resulted from glycosylation. The IL-20-flag-fusion protein was secreted as a single band, with a molecular weight of about 18 kilodaltons. This suggests that the protein was not glycosylated.

When the chemiluminescence signals were quantified, this showed that IL-19 and IL-22 were produced at similar levels. In contrast, IL-20 and mda-7 were produced at levels 7 fold less.

### Example 2

The supernatants of the HEK-293 transfectants, described supra were used to assess the interaction of the cytokines with class II cytokine receptors.

The cell line HT-29 endogenously expresses IL-22R and IL-10Rβ. Samples of these cells were transfected with construct “pGRR5.” This construct, which contains 5 copies of the STAT binding site of the FcγRI gene, inserted upstream of a luciferase gene controlled by the TK promoter, can be used to monitor STAT activation by IL-22. It was assumed that, if the mechanism of action for the other IL-22 homologues was the same, a similar assay could be used to detect it.

Samples of HT-29 were electroporated (10⁷ cells in 400 μl, 250V, 192Ω, 1,200 μF), with 15 μg of pGRR5. The cells were seeded in 96 well plates, at 10⁵ cells/well, incubated for 5 hours at 37° C., and then were either preincubated, or not, for 1 hour with either anti-IL22R antiserum (1/500), or anti-IL-10Rβ antibodies (6 μg/ml). The cells were then stimulated, for two hours, with the different supernatants. Luciferase activity was measured, using commercially available materials.

The anti-hIL22R antibodies were made by transfecting P815 mastocytoma cells with cDNA encoding human IL-22R that had been incorporated into plasmid pEF-BOS puro. To elaborate, IL-22R cDNA was amplified from a hepatoma cell line, i.e., “Hep G2,” using sense primer:

ggaggactag ttgccagccc cgatgagga

(SEQ ID NO: 6). This sense primer contains a restriction site for Spe1. An antisense primer containing a restriction site for NotI, i.e.:

gtgtggcggc cgcaggcatg ggattgacag c

(SEQ ID NO: 7) was also used. These primers facilitate direct cloning into the pEF-BOS puro expression vector. See Demoulin, et al., Mol. Cell Biol. 16:4710 (1996), incorporated by reference. The transfectants were injected into DBA/2 mice. After the rejection of the tumors by the mice, their resulting sera contained high titers of neutralizing anti-hIL-22 antibodies. This is the antiserum referred to supra.

The HT-29 cells were responsive to IL-22, but not to the other cytokines.

### Example 3

The work of example 2 was extended, by cotransfecting the HT-29 cells with pGRR5, and cDNA (15 μg), for either IL-20Rβ or IL-20Rα. In all other ways, the experiment was exactly the same as in example 2.

When the HT-29 cells were transfected with IL-20Rβ cDNA, both mda-7 and IL-20 induced luciferase production; however, this effect was blocked completely by anti-IL-22R antiserum, indicating formation of a previously unobserved IL-20R, which contains IL-22R and IL-20Rβ.

When the HT-29 cells were transfected with cDNA for both IL-20Rα and IL-20Rβ, they became receptive to all of mda-7, IL-20, and IL-19. Further, luciferase production was not affected by the anti-IL-22R antibodies, indicating that the activity was independent of the chain.

Transfection with IL-20Rα cDNA alone did not provoke any response.

These results show that IL-20Rβ is required for the described process.

### Example 4

A further set of experiments were then carried out, in order to characterize the different types of receptor complexes further.

HEK-293 cells express IL-10Rβ endogenously, but do not express IL-22R. Untransfected cells were admixed with supernatants, as described supra, and did not respond to any of the homologues. Samples of HEK-29 cells were seeded in 24 well plates, at 2×10⁵ cells/well one day before transfection, and were then transfected, using 100 ng of pCGG5, and 500 ng of plasmid encoding IL-20Rα, IL-20Rβ, or IL-22R, using the lipofectamine method referred to in example 1 and assayed with supernatants, as described supra. The cDNA for IL-20Rα was obtained by amplification of a placenta library. A cDNA fragment encoding the extracellular domain was amplified using:

gccggatcca tgcggccgct gccgctgccg

(SEQ ID NO: 8), and

atcgctagcc atttagcctt gaactctgat g

(SEQ ID NO: 9),

digested with BamHI restriction endonuclease, and cloned into BamHI and EcoRV sites of commercially available, pcDEF3 vector. The resulting plasmid is referred to as “pEF2-CRF2-8EC.” The PCR product encoding the transmembrane and intracellular domains was generated using:

gtggctagcc tggtatgttt tgcccat

(SEQ ID NO: 10), and

gcgaattcgt ctggcaaaca tttattga

(SEQ ID NO: 11), which contain NheI and EcoRI sites, respectively, to facilitate cloning into pEF2-CRF2-8EC.

The cDNA for IL-20Rβ was amplified from K562 leukemia cells, using:

ttggctagca acaatgttct aggtc

(SEQ ID NO: 12), and

tgggcgcggc cgcaaaccta tgagat

(SEQ ID NO: 13), which contain NheI and NotI sites, for cloning into pCEP4.

When IL-22R cDNA was transfected into the cells, IL-22 induced luciferase production and STAT-3 phosphorylation, but none of the homologues did. Cells transfected with both IL-22R and IL-20Rβ responded to IL-22, IL-20 and mda-7. Transfection with IL-20Rβ alone did not confer any responsiveness. When both IL-20Rα and IL-20Rβ were transfected, all of the mda-7, IL-19 and IL-20 provoked responses, but IL-22 did not. There was no response when IL-20Rα was used alone.

In each of these experiments, luciferase induction correlated with STAT-3 phosphorylation.

### Example 5

The experiments set forth, supra, showed that two different complexes bound with IL-20 and mda-7. This begged the question of whether one complex would respond more favorably to one of the cytokines.

In order to examine this question, HT-29 cells were transfected, either with IL-20Rβ alone, or both IL-20Rβ and IL-20Rα, in the same way described supra. Varying dilutions (10%, 1%, and 0.1%) of supernatants were used. Luciferase activity was measured two hours after stimulation.

When both IL-20Rα and IL-20Rβ were transfected into cells, both mda-7 and IL-20 dilutions showed a similar dose-response curve, indicating similar sensitivity to both cytokines. When only IL-20Rβ was transfected, the HT-29 cells showed better responsiveness to mda-7 at non-saturating dilutions (1% and 0.1% supernatant), indicating that this type of complex is more sensitive to mda-7.

Parallel results were obtained when experiments were repeated, using HEK-293 cells.

### Example 6

The experiments set forth, supra, showed that IL-22R can associate both with IL-10Rβ and IL-20Rβ, suggesting that complexes of IL-20Rβ and IL-22R could mediate an IL-22response. Experiments to test this possibility had to account for the fact that IL-10Rβ is ubiquitously expressed. Hence, the role of IL-10Rβ was assessed with an anti-IL-10Rβ antibody. HT-29 cells were transfected with pGRR5, and IL-20Rβ cDNA, as described supra. The transfectants were cultured, also as described, supra, and were then incubated with 10% supernatant from HEK-293 cells that had either been transfected with IL-22 cDNA, or had been mock transfected. Luciferase activity was monitored 2 hours later.

The results showed that the anti-IL-10Rβ antibody blocked IL-22 activity both in control cells, and in cells transfected with IL-20Rβ cDNA. This indicates that IL-20Rβ cannot substitute for IL-10Rβ, when this is not accessible to IL-22.

In related experiments, the antibody did not affect activity of mda-7 or IL-20, in the same cells.

### Example 7

Work by Dumoutier, et al., J. Immunol 166:7090(2001) and Kotenko, et al., J. Immunol 166:7096(2001), both of which are incorporated by reference, showed that IL-22BP binds to IL-22. There have not been any other reports on whether this molecule binds to other cytokines. As it exhibits the same degree of homology with the extracellular domains of IL-22R and IL-20Rα suggested that IL-22BP could also bind IL-20, or other molecules.

To test this, first maleic anhydride activated polystyrene plates were coated with biotinylated anti-flag antibody by incubating the plates, overnight, at 4° C., with 12.5 μg/ml of the antibody in PBS. Plates were washed in PBS buffer containing Tween 20 (0.01%), and then blocked with bovine serum albumin (1% in PBS) for two hours, followed by incubation for 2 hours with 50 μl of cytokine flag fusion proteins, by way of HBEK-293 supernatants, produced as described supra. After washing, 10% supernatants of cells that had been transfected with vectors that produced IL-22BP-Ig were added. See Dumoutier, et al., supra, incorporated by reference, for information on production of this fusion protein. The supernatant was removed after 2 hours, and any bound IL-22BP-Ig was detected by adding anti-mouse IgG3 polyclonal antibodies, coupled to peroxidase. The enzymatic activity was then determined by adding 3, 3′, 5, 5′ tetramethylbenzidine. The reaction was stopped by adding 20 μl of H₂SO₄ (2M). Reactivity was determined by reading absorbance values, at 450 nm.

Only IL-22 was able to bind IL-22BP-Ig. No homologues showed any activity.

In related experiments, the inhibitory effect of IL-10 homologues on the binding of IL-22BP to IL-22 was determined. To do this, plates, as described supra, were coated with recombinant human IL-22, as described by Dumoutier, et al., Proc. Natl. Acad. Sci USA 97:10144(2000), incorporated by reference. The plates were then contacted with IL-22BP-Ig (10% supernatant) that had been preincubated with the various IL-10 homologues, 2 hours before contact with the plates. Any bound IL-22BP-Ig was determined in the same manner described in the first set of experiments. The interaction between IL-22 and IL-22BP was detectable with the anti-Ig antibody, and only IL-22 supernatants were able to block the IL-22BP binding.

The foregoing examples describe, inter alia, a new cytokine receptor complex, which comprises an IL-22R molecule, and an IL-20Rβ molecule. As is shown, supra, this receptor complex facilitates the binding of cytokines such as mda-7 and IL-20. The examples also show that complexes containing IL-20Rα and IL-20Rβ bind these cytokines, as well as IL-19. Both complexes can serve, inter alia, as agents for determining whether or not their respective binding partners are present in a sample. For example, it is known that IL-20Rα and IL20Rβ form a receptor for IL-20, and also for mda-7 and IL-19. Complexes of IL-22R and IL-20Rβ form a receptor for mda-7 and IL-19, but not IL-20. Hence, by using various combinations of these receptors, one can determine which IL-10 related cytokines are or are not present in a sample.

As noted, supra, transgenic mice experiments establish that IL-20 is associated with neonatal lethality, and various skin abnormalities. Hence, one can, e.g., inhibit the effect of IL-20 on receptive cells by contacting the cells with other cytokines which compete for binding with IL-20, or by contacting the receptor with, e.g., an inhibitor of receptor binding, such as an antibody, or some other form of inhibiting molecule. Exemplary of such antagonists or inhibitors are, e.g., mda-7 or IL-19 mutants which have been mutated to abolish activity without abolishing their affinity for the receptor, soluble binding partners for IL-20, such as soluble, extracellular fragments of IL-20 receptors, and so forth.

This mechanism provides an approach to therapies where proliferation of epidermal cells, such as skin cells, is contraindicated. Exemplary of such conditions are atopic dermatitis, psoriasis, seborrhoeic keratitis, keratodermas and other conditions involving excess proliferation of epidermal and/or skin cells. In such therapeutic approaches, one administers to a subject in need thereof an amount of an agent which binds to IL-20 thereto. Such agents can be any of the inhibitors described supra, including antibodies, portions of antibody molecules which bind to the IL-20 receptor or receptors, fragments of IL-20 itself which are capable of binding to the receptor but do not have effector function, mutated forms of IL-20 which have not lost receptor affinity but are inactive. Such forms of molecules are known for other interleukins, soluble forms of IL-20 receptors, and so forth.

This observation, i.e., that IL-20 stimulates proliferation of epidermal cells, such as skin cells, permits the skilled artisan to develop assays to determine if a test substance of interest has an inhibitory effect on the proliferation of such cells. In effect, one combines the substance of interest with IL-20, and epidermal cells, such as skin cells, determines proliferation, and compares the result to the use of IL-20 in the system alone. A decrease in proliferation indicates an inhibitory effect of the test substance. One can further characterize the specific activity of the test substance by performing parallel experiments using a cell sample wherein the cells present complexes of IL-22R and IL-20Rβ on their surface, and where complexes of IL-20α and IL-20Rβ are presented. For example, if there is inhibition, following stimulation of cells with a test substance and IL-20 and the values are equal, then the inhibitor would probably act on either IL-20 itself, or IL-20Rβ. If the inhibition is greater in the cells presenting IL-22R/IL-20Rβ complexes than those presenting IL-20Rα/IL-20Rβ complexes, then the inhibitor probably acts through IL-22R, and vice versa.

Similarly, the observation that IL-20 responds to complexes of IL-22R and IL-20Rβ suggests methods for enhancing or providing for binding of IL-20 to cells. This can be accomplished, e.g., by transfecting the target cells with nucleic acid molecules encoding IL-20Rβ, in quantities sufficient to form additional, “type II” receptor complexes. As was pointed out, supra, IL-22 is known to have specific effects on cells. One can enhance these effects by, e.g., transforming or transfecting target cells so that they present a greater number of IL-22R and IL-20Rβ. Hence, if the target cells do express Il-22R but not IL-20Rβ, one can transform or transfect the cells with vectors that express Il-20Rβ, to generate additional receptors. Alternatively, one can transform or transfect the cells with nucleic acid molecules encoding both of these molecules, or constructs encoding fusion proteins of the two molecules, one complete molecule and the binding portion of the other, or two binding portions. The relevant portions or molecules could be joined via, e.g., a linker sequence or some other construct which facilitates proper folding and functionality of the molecule.

Another aspect of the invention is the modulation of the effect of the cytokine referred to as interleukin-9 (“IL-9”). It is known that IL-9 induces expression of IL-22, and that IL-9 is associated with, inter alia, asthma, atopic allergies, gut inflammation, induction of IgE and inhibition of IgG. One can mediate the effect of IL-9 indirectly, by modulating the ability of IL-22 to bind to a target receptor. The methodologies for modulating the binding are set forth, supra, and need not be reiterated here.

The results also indicate that, by appropriate selection of transfectants, one can “type” samples, such as samples taken from patients, to determine if particular cytokines are present, and to quantify these. For example, it was shown, supra that complexes of IL-20Rα and IL-20Rβ bind to all of IL-19, IL-20, and mda-7. Hence, in a first part of an assay, one contacts a sample of interest with cells which present complexes of IL-20Rα and IL-20Rβ, and determine if any binding occurs. If binding does occur, then one of these cytokines is present. In a second step, one can then assay the sample with complexes of IL-20Rβ and IL-22R. If the results of this assay are negative, then it can be concluded that IL-19 is present in the sample, but not IL-20 or mda-7.

Similarly, if both assays are positive, one can conclude that either or both of mda-7 and IL-20 are present in the sample. Further definition of the sample content can be ascertained by comparing binding of the materials to IL-22R/IL-20Rβ complexes, and IL-20Rα/IL-20Rβ complexes, because the former bind mda-7 better than the latter.

Other aspects of the invention will be clear to the skilled artisan, and need not be presented in further details.

We claim:

1. An isolated complex comprising an interleukin-22 receptor molecule, and an interleukin-20 receptor β molecule.

2. The isolated complex of claim 1, wherein each of said molecules are mammalian molecules.

3. The isolated complex of claim 2, wherein each of said molecules are human molecules.

4. A method for inhibiting effect of interleukin-22 on a cell, comprising contacting said cell with a molecule which inhibits interaction of interleukin-10 receptor β molecules with interleukin 22 receptor molecules, in an amount sufficient to inhibit said interaction.

5. The method of claim 4, wherein said molecule is an antibody.

6. The method of claim 4, wherein said cell is a cell of a patient suffering from an interleukin-9 associated disorder.

7. The method of claim 6, wherein said disorder is asthma, an atopic allergy, excess IgE production, gut inflammation, or insufficient IgG production.

8. The method of claim 4, wherein said molecule is a soluble form of one of interleukin-10 receptor β or interleukin-22 receptor.

9. The method of claim 4, wherein said molecule is a mutant of IL-19 or a mutant of mda-7, wherein said mutant of IL-19 or mutant of mda-7 retains receptor affinity but has lost activity.

10. A method for inhibiting effect of interleukin-20 (IL-20) on a cell, comprising contacting said cell with at least one inhibitor molecule selected from the group consisting of (i) an inhibitor of IL-22R, (ii) an inhibitor of IL-20Rα, (iii) an inhibitor of IL-20Rβ, and (iv) an inhibitor of a complex of IL-22R and IL-20Rβ, in an amount sufficient to inhibit binding of IL-20 to said cell.

11. The method of claim 10, wherein said cell is a skin cell.

12. The method of claim 10, wherein said inhibitor is an antibody.

13. The method of claim 10, comprising contacting a cell of a subject suffering from a condition characterized by inappropriate proliferation of skin cells.

14. The method of claim 13, wherein said condition is atopic dermatitis, psoriasis, seborrhoeic keratitis, a neoplasm, or a keratoderma.

15. A method for determining if a substance has epidermal cell proliferation inhibition activity, comprising admixing a sample of epidermal cells which present an IL-20 and a substance to be tested, measuring epidermal cell proliferation, and comparing said proliferation to proliferation resulting from admixing a sample of said epidermal cells with IL-20 alone, a decrease in proliferation being indicative of epidermal cell proliferation inhibition activity of said substance.

16. The method of claim 15, wherein said epidermal cells are skin cells.

17. A method for inhibiting effect of interleukin 22 on a cell, comprising contacting said cell with a molecule which inhibits interaction of interleukin 22 receptor molecules and interleukin 20 receptor β molecules, in an amount sufficient to inhibit said interaction.

18. The method of claim 17, wherein said cell is a cell of a patient suffering from an interleukin-9 associated disorder.

19. The method of claim 18, wherein said disorder is asthma, an atopic allergy, excess IgE production, gut inflammation, or insufficient IgG production.

20. A method for identifying a molecule which modulates activity of IL-20 or mda-7, comprising admixing a cell which expresses IL-22R and IL-Rβ with said molecule and one of IL-20 and mda-7, determining effect of said IL-20 or mda-7 on said cell in the absence of said molecule to determine if said molecule modulates activity of said Il-20 or mda-7.

21. A method for identifying a molecule which modulates activity of IL-19 or mda-7, comprising admixing a cell which expresses IL-20Rα and IL-20Rβ with said molecule and one of IL-19 or mda-7, determining effect of said IL-19 or mda-7 on said cell, and comparing effect of said Il-19 or mda-7 on said cell in the absence of said molecule to determine if said molecule modulates activity of IL-19 or mda-7.

22. A method for inhibiting effect of at least one of mda-7 and IL-19 on a cell, comprising contacting said cell with a molecule which inhibits interaction of IL-20Rα and IL-20Rβ, in an amount sufficient to inhibit said interaction.

23. The method of claim 22, wherein said molecule is an antibody.

24. The method of claim 22, wherein said molecule is a soluble form of IL-20Rα or a soluble form of IL-20Rβ.

25. The method of claim 22, wherein said molecule is a mutant form of IL-20 which retains affinity for a complex of Il-20Rα and IL-20Rβ, but has lost activity.
